(19) United States
(12) Patent Application Publication
(10) Pub. No.: US 2024/0324941 A1
(43) Pub. Date: Oct. 3, 2024

(54) SYSTEM AND METHOD OF IDENTIFYING AND MONITORING NEURAL SYNCHRONY

(71) Applicant: Christine Ramos, East Rockaway, NY (US)

(72) Inventor: Christine Ramos, East Rockaway, NY (US)

(21) Appl. No.: 18/140,690

(22) Filed: Apr. 28, 2023

Related U.S. Application Data
(60) Provisional application No. 63/455,009, filed on Mar. 28, 2023.

(51) Int. Cl. A61B 5/374; A61B 5/00; A61B 5/0205; A61B 90/00

(52) U.S. Cl. CPC A61B 5/374; A61B 5/0205; A61B 5/6803; A61B 5/7285; A61B 5/743; A61B 90/361

(57) ABSTRACT

A system for identifying neural synchrony includes first and second EEG headsets configured to detect neural activity in first and second users, respectively. A computer is in communication with the first and second EEG headsets. The computer is configured to receive a first data set from the first EEG headset. The first data set includes data of the neural activity of the first user. The computer is configured to receive a second data set from the second EEG headset. The second data set includes data of the neural activity of the second user. The computer is configured to compare the first data set with the second data set. The computer is configured to identify an occurrence of neural synchrony between the first user and the second user based on the comparison of the first data with the second data set.

### CROSS-REFERENCE TO RELATED APPLICATION

The present application claims priority to U.S. Provisional Patent Application No. 63/455,009, filed on Mar. 28, 2023, the entire contents of which are incorporated by reference herein.

### FIELD

The present disclosure relates to neural synchrony, and more particularly, to a system and method of identifying and monitoring neural synchrony.

### BACKGROUND

Neural synchrony refers to the correlation of brain activity across at least two people. In social and affective neuroscience, neural synchrony refers particularly to the degree of similarity between the spatio-temporal neural fluctuations of at least two people. This phenomenon represents the convergence and coupling of different individual's neurocognitive systems. Neural synchrony is hypothesized to be the neural substrate for many forms of interpersonal dynamics and shared experiences. Some research refers to neural synchrony as, for example, inter-brain synchrony, brain-to-brain coupling, inter-subject correlation, between-brain connectivity, or neural coupling. In some literature, neural synchrony is notably distinct from intra-brain synchrony, which refers to the coupling of activity across regions of a single individual's brain.

### SUMMARY

Provided in accordance with aspects of the present disclosure is a system for identifying and monitoring neural synchrony including a first electroencephalogram (EEG) headset including a number of first electrodes. The first EEG headset is configured to be worn by a first user. The first electrodes of the first EEG headset are configured to detect neural activity in the first user. A second EEG headset includes a number of second electrodes. The second EEG headset is configured to be worn by a second user. The second electrodes of the second EEG headset are configured to detect neural activity in the second user. An image capture device is configured to capture first images of a first behavior performed by the first user and second images of a second behavior performed by the second user. A computer is in communication with the first EEG, the second EEG, and the image capture device. The computer includes a processor and a memory. The memory is configured to store computer instructions configured to instruct the processor to receive a first data set from the first EEG headset. The first data set includes data of the neural activity of the first user. The processor is instructed by the computer instructions to receive a second data set from the second EEG headset. The second data set includes data of the neural activity of the second user. The processor is instructed by the computer instructions to compare the first data set with the second data set. The processor is instructed by the computer instructions to identify an occurrence of neural synchrony between the first user and the second user based on the comparison of the first data with the second data set.

In an aspect of the present disclosure, the computer is instructed to receive a third data set from the image capture device. The third data set includes data of the first images of the first behavior performed by the first user. The computer is instructed to receive a fourth data set from the image capture device. The fourth data set includes data of the second images of the behavior performed by the second user. The computer is instructed to compare the third data set with the fourth data set. The computer is instructed to identify an occurrence of a joint behavior performed by each of the first user and the second user based on the comparison of the third data set with the fourth data set.

In an aspect of the present disclosure, the first and second data sets are each captured during a same predetermined time period as each other. The first data set is compared with the second data set with respect to the same predetermined time period.

In an aspect of the present disclosure, the third and fourth data sets are each captured during the same predetermined time period. The third data set is compared with the fourth data set with respect to the same predetermined time period.

In an aspect of the present disclosure, the first EEG headset is configured to be worn by a child, and the second EEG headset is configured to be worn by a parent of the child.

In an aspect of the present disclosure, the first electrodes or the second electrodes are configured to capture beta waves, alpha waves, theta waves, delta waves, gamma waves, and/or epsilon waves.

In an aspect of the present disclosure, the first data set includes data of beta waves, alpha waves, theta waves, delta waves, gamma waves, and epsilon waves of the first user, and the second data set includes data of beta waves, alpha waves, theta waves, delta waves, gamma waves, and epsilon waves of the second user. The computer instructions instruct the processor to compare the data of the beta waves, alpha waves, theta waves, delta waves, gamma waves, and epsilon waves of the first user with the data of the beta waves, alpha waves, theta waves, delta waves, gamma waves, and epsilon waves of the second user. The computer instructions instruct the processor to identify the occurrence of neural synchrony between the first user and the second user based on the comparison of the beta waves, alpha waves, theta waves, delta waves, gamma waves, and epsilon waves of the first user with the beta waves, alpha waves, theta waves, delta waves, gamma waves, and epsilon waves of the second user.

In an aspect of the present disclosure, the computer is instructed to identify intuitive interbrain synchrony, neural entrainment, motor-induced neural synchrony, attention enhanced neural synchrony, and/or stimuli-based interbrain synchrony.

In an aspect of the present disclosure, the image capture device is a digital camera.

In an aspect of the present disclosure, a first display is configured to receive the first data set from the first EEG headset. The first display is configured to display a first visual representation of brain wave data of the first user. A second display is configured to receive the second data set from the second EEG headset. The second display is configured to display a second visual representation of brain wave data of the second user.

In an aspect of the present disclosure, the first display is arranged to display the first visual representation of brain wave data to the first user, and the second display is arranged to display the second visual representation of brain wave data to the second user.

In an aspect of the present disclosure, the first display is arranged to display the first visual representation of brain wave data to the second user, and the second display is arranged to display the second visual representation of brain wave data to the first user.

In an aspect of the present disclosure, a first monitoring device is configured to detect pulse rate, oxygen saturation, cardiac cycle, and/or respiration rate of the first user. A second monitoring device is configured to detect pulse rate, oxygen saturation, cardiac cycle, and/or respiration rate of the second user. The first monitoring device and the second monitoring device are in communication with the computer to deliver data from the first monitoring device and the second monitoring device to the computer.

In an aspect of the present disclosure, each of the first monitoring device and the second monitoring device includes a pulse oximeter, an electrocardiography device, a photoplethysmography device, and/or a breath monitoring device.

Provided in accordance with aspects of the present disclosure is a computer-implemented method of identifying neural synchrony including receiving a first data set from a first EEG headset including a number of first electrodes. The first EEG headset is configured to be worn by a first user. The first electrodes of the first EEG headset are configured to detect neural activity in the first user. The first data set includes data of the neural activity of the first user. The computer-implemented method includes receiving a second data set from a second EEG headset including a number of second electrodes. The second EEG headset is configured to be worn by a second user. The second electrodes of the second EEG headset are configured to detect neural activity in the second user. The second data set includes data of the neural activity of the second user. The computer-implemented method includes comparing the first data set with the second data set. The computer-implemented method includes identifying an occurrence of neural synchrony between the first user and the second user based on the comparison of the first data with the second data set.

### BRIEF DESCRIPTION OF THE DRAWINGS

Various aspects and features of the present disclosure are described hereinbelow with reference to the drawings wherein:

FIG. 1 is a schematic diagram of a system for identifying and monitoring neural synchrony according to aspects of the present disclosure;

FIG. 2 is a schematic diagram of another system for identifying and monitoring neural synchrony according to aspects of the present disclosure;

FIG. 3A is a conceptual illustration of parent-child behavioral synchrony according to aspects of the present disclosure;

FIG. 3B is a conceptual illustration of parent-child neural synchrony according to aspects of the present disclosure;

FIG. 4 is a conceptual illustration of in-phase neural synchrony, phase-lagged neural synchrony and an absence of neural synchrony according to aspects of the present disclosure;

FIG. 5A is a conceptual illustration of intuitive formulation of interbrain synchrony according to aspects of the present disclosure;

FIG. 5B is a conceptual illustration of neural entrainment according to aspects of the present disclosure;

FIG. 5C is a conceptual illustration of motor-induced neural synchrony according to aspects of the present disclosure;

FIG. 5D is a conceptual illustration of attention-enhanced neural synchrony according to aspects of the present disclosure;

FIG. 5E is a conceptual illustration of interbrain neural synchrony according to aspects of the present disclosure;

FIG. 6 is a flowchart of a method of identifying and monitoring neural synchrony according to aspects of the present disclosure;

FIG. 7 is a flowchart of a method of identifying and monitoring neural synchrony including comparing behaviors performed by users according to aspects of the present disclosure; and

FIG. 8 is a block diagram of an exemplary computer for implementing the method of identifying and monitoring neural synchrony according to aspects of the present disclosure.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

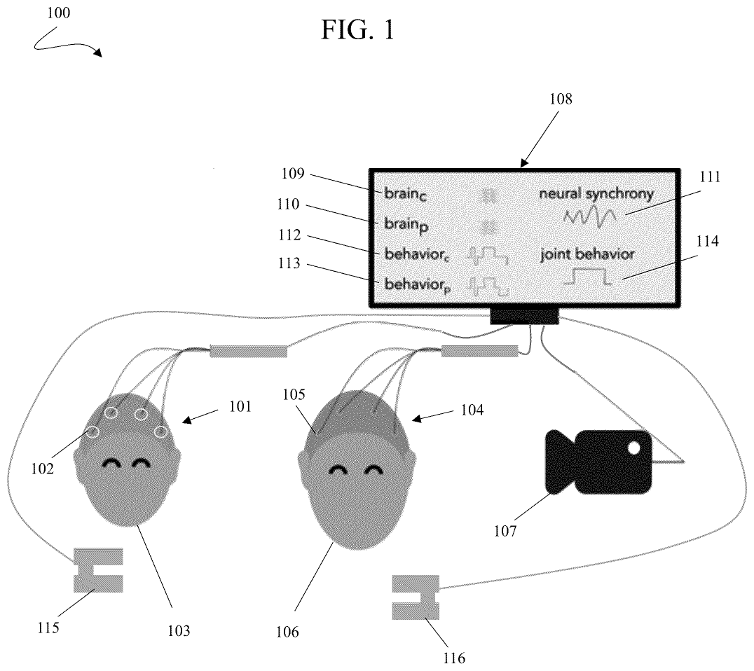

FIG. 1 is a schematic diagram of a system 100 for identifying and monitoring neural synchrony. Referring particularly to FIG. 1, the system 100 for identifying and monitoring neural synchrony includes a first electroencephalogram (EEG) headset 101 including a number of first electrodes 102. The first EEG headset 101 is configured to be worn by a first user 103. The first electrodes 102 of the first EEG headset 101 are configured to detect neural activity in the first user 103.

A second EEG headset 104 includes a number of second electrodes 105. The second EEG headset 104 is configured to be worn by a second user 106. The second electrodes 105 of the second EEG headset 104 are configured to detect neural activity in the second user 106.

The first electrodes 102 and/or the second electrodes 105 may be an array of electrodes arranged to be positioned adjacent various anatomical regions of a user's head. The first electrodes 102 and/or the second electrodes 105 may be included in a microelectrode array (MEA), which may be referred to as a multielectrode array including multiple (e.g., tens to thousands) microelectrodes through which neural signals are obtained or delivered. The first electrodes 102 and/or the second electrodes 105 may measure electrical current or voltage generated in a user's brain. In an aspect of the present disclosure, the first electrodes 102 and/or the second electrodes 105 may be configured to operate in a bidirectional fashion such that they can also be used to provide a stimulating pattern of electric current or voltage.

An image capture device 107 is configured to capture first images of a first behavior performed by the first user 103 and second images of a second behavior performed by the second user 106. The behaviors may be recorded using one or more still images, numerous still images, or as video recordings. As an example, the video recordings may be analyzed by a classifier, as described herein, to detect distinct behaviors performed by the first user 103 and/or the second user 106. The video recordings may include time stamp data to determine if overlapping behaviors performed by the first user 103 and the second user 106 occur simultaneously, substantially simultaneously, in a phase-lagged manner, or at distinct time intervals.

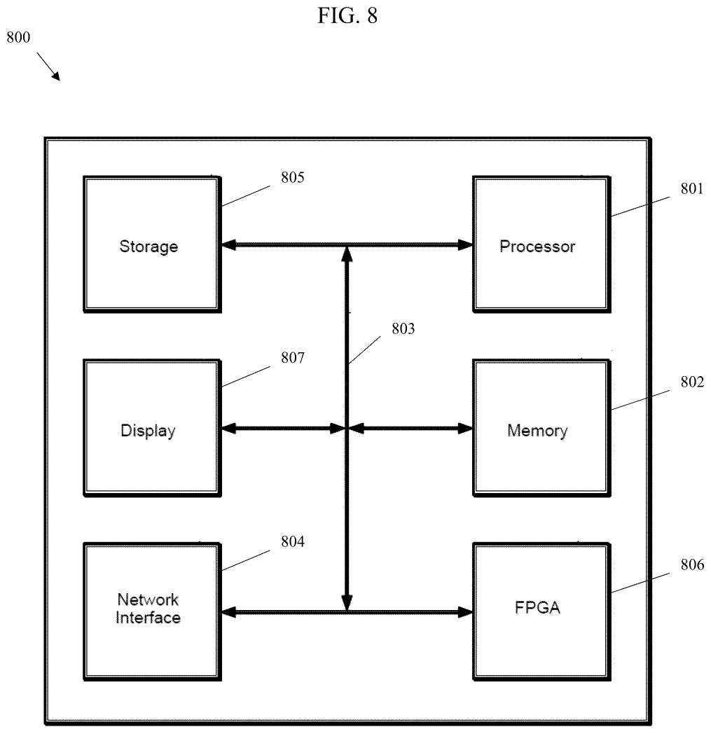

A computer 108 is in communication with the first EEG headset 101, the second EEG headset 104, and the image capture device 107. The computer 108 includes a processor (see, e.g., processor 801 described with reference to FIG. 8) and a memory (see, e.g., memory 802 described with reference to FIG. 8). The memory is configured to store computer instructions configured to instruct the processor to receive a first data set 109 from the first EEG headset 101. The first data set 109 includes data of the neural activity of the first user 103. The processor is instructed by the computer instructions to receive a second data set 110 from the second EEG headset 104. The second data set 110 includes data of the neural activity of the second user 106. The processor is instructed by the computer instructions to compare the first data set 109 with the second data set 110. The processor is instructed by the computer instructions to identify an occurrence of neural synchrony 111 between the first user 103 and the second user 106 based on the comparison of the first data set 109 with the second data set 110.

In an aspect of the present disclosure, the image capture device 107 is a digital camera. The image capture device 107 may be configured to capture at least one of still images, video images, or audio recordings. The image capture device 107 may be a camera integrated into or connected with a smartphone, tablet computer, or laptop computer.

The first EEG headset 101 and/or the second EEG headset 104 may be dry EEG headsets. For example, the first EEG headset 101 and/or the second EEG headset 104 may be an EEG headband. The EEG headband may be wearable headband configured to stretch around the user's head to prevent movement of the EEG headband, such as during use. The first EEG headset 101 and/or the second EEG headset 104 may also be an EEG headset including at least some rigid components, or a combination of rigid components and various flex points connecting the rigid components with each other to contour around a user's head. As an example, a child may wear an EEG headband, and a parent or adult (e.g., caregiver of the child) may wear an EEG headset.

In an aspect of the present disclosure, the computer 108 is instructed to receive a third data set 112 from the image capture device 107. The third data set 112 includes data of the first images of the first behavior performed by the first user 103. The computer 108 is instructed to receive a fourth data set 113 from the image capture device 107. The fourth data set 113 includes data of the second images of the behavior performed by the second user 106. The computer 108 is instructed to compare the third data set 112 with the fourth data set 113. The computer 108 is instructed to identify an occurrence of a joint behavior 114 performed by each of the first user 103 and the second user 106 based on the comparison of the third data set 112 with the fourth data set 113.

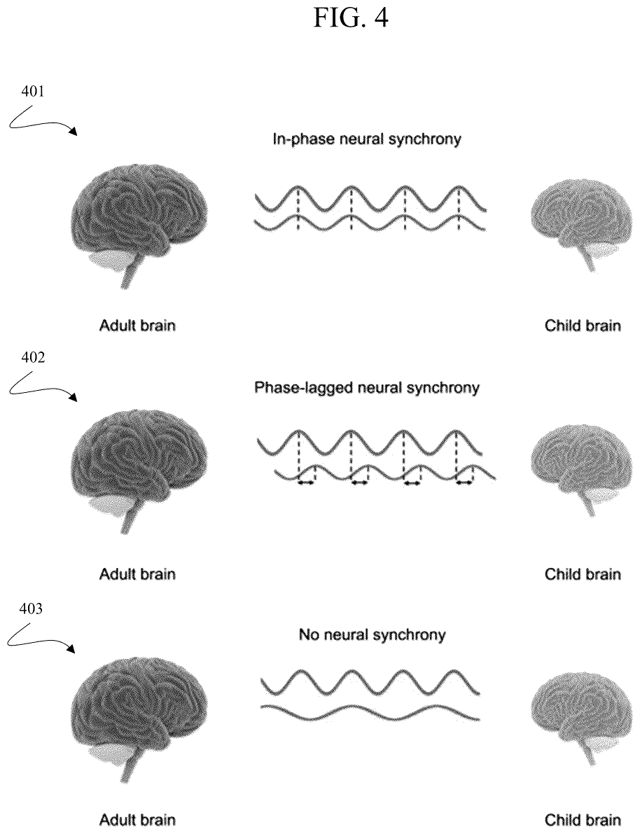

The first and second data sets 109 and 110 may each be captured during the same predetermined time period as each other. The first data set 109 is compared with the second data set 110 with respect to the same predetermined time period. Timing data can be used to determine the degree to which neural synchrony is occurring in-phase (see, e.g., 401 FIG. 4) or in a phase-lagged manner (see, e.g., 402 FIG. 4), or if neural synchrony is not occurring (see, e.g., 403 FIG. 4).

The third and fourth data sets 112 and 113 may each be captured during the same predetermined time period as each other. The third data set 112 is compared with the fourth data set 113 with respect to the same predetermined time period. Timing data can be used to determine the degree to which behavioral synchrony is occurring in-phase or in a phase-lagged manner, or if behavioral synchrony is not occurring (see, e.g., FIG. 4).

The first, second, third, and fourth data sets 109, 110, 112, and 113 may each be captured during a same time period as each other to determine if both neural synchrony and behavioral synchrony are each occurring (e.g., in an in-phase or phase-lagged manner).

In an aspect of the present disclosure, the first electrodes 102 or the second electrodes 105 are configured to capture beta waves, alpha waves, theta waves, delta waves, gamma waves, and/or epsilon waves.

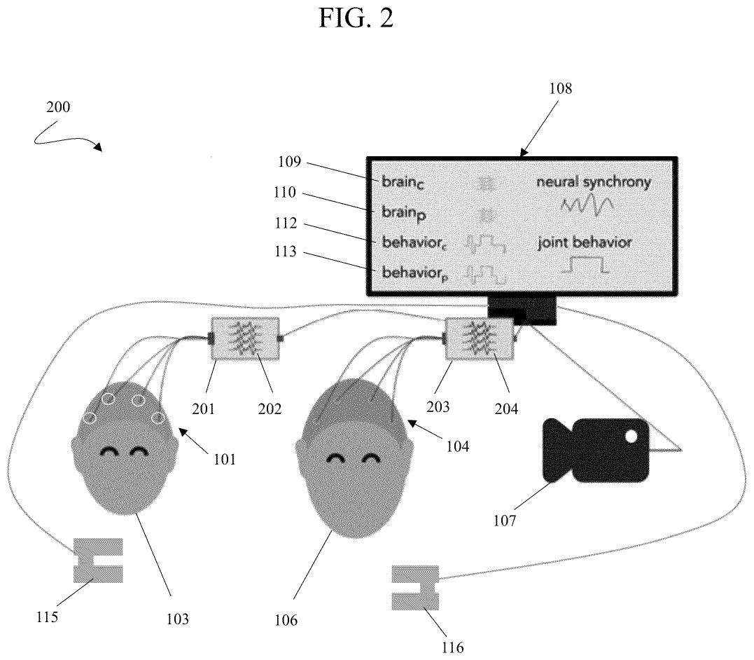

Unless otherwise indicated below, the system 200 described with reference to FIG. 2 is substantially the same as the system 100 described above with reference to FIG. 1, and thus duplicative descriptions may be omitted below.

FIG. 2 is a schematic diagram of a system 200 for identifying and monitoring neural synchrony. Referring particularly to FIG. 2, a first display 201 is configured to receive the first data set 109 from the first EEG headset 101. The first display 201 is configured to display a first visual representation 202 of brain wave data of the first user 103. A second display 203 is configured to receive the second data set 110 from the second EEG headset 104. The second display 203 is configured to display a second visual representation 204 of brain wave data of the second user 106.

The displays 201 and 204 may communicate with the computer 108 di-directionally, and thus the displays 201 and 204 may transmit data to computer 108 or may receive instructions from the computer 108. For example, the computer 108 may provide instructions on the first visual representation 202 of brain wave data of the first user 103 and/or the second visual representation 204 of brain wave data of the second user 106.

In an aspect of the present disclosure, the first display 201 is arranged to display the first visual representation 202 of brain wave data to the first user 103, and the second display 203 is arranged to display the second visual representation 204 of brain wave data to the second user 106.

In an aspect of the present disclosure, the first display 201 is arranged to display the first visual representation 202 of brain wave data to the second user 106, and the second display 203 is arranged to display the second visual representation 204 of brain wave data to the first user 103.

In an aspect of the present disclosure, the first display 201 is arranged to display the first visual representation 202 of brain wave data and the second display 203 is arranged to display the second visual representation 204 of brain wave data to the same user (e.g., to one of the first user 103 or the second user 106, but not to the other of the first user 103 or the second user 106). For example, the displays 201 and 203 may be arranged to display all visual data to a parent or caregiver, but not to a child of the parent or caregiver.

As an example, the first display 201 and/or the second display 203 may be displays of a smartphone, tablet, or laptop computer. The first display 201 and/or the second display 203 may be a monitor, such as a computer monitor, or a television monitor, such as a flat-screen television monitor. The first display 201 and/or the second display 203 may be incorporated into a single screen. For example, a single monitor or display device may display the content of each of the first display 201 and the second display 203 in a split screen format.

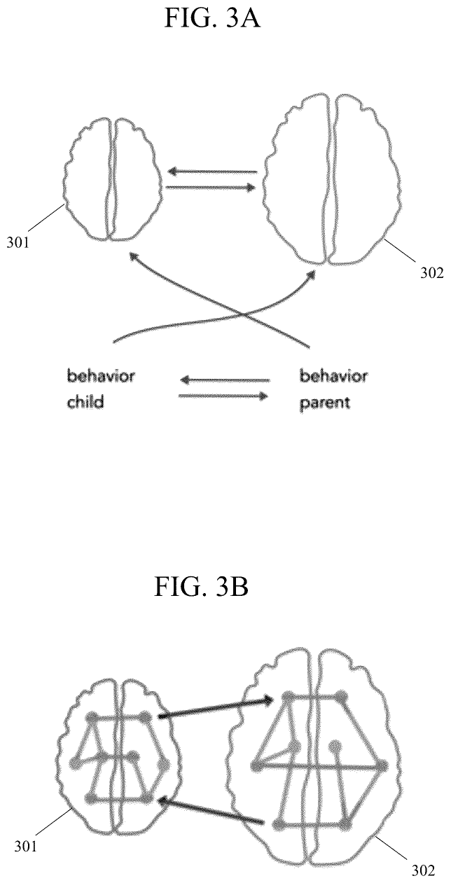

Referring particularly to FIGS. 3A to 5E, the first data set 109 includes data of beta waves, alpha waves, theta waves, delta waves, gamma waves, and epsilon waves formed in the brain 301 (see, e.g., FIG. 3A) of the first user 103, and the second data set 110 includes data of beta waves, alpha waves, theta waves, delta waves, gamma waves, and epsilon waves formed in the brain 302 (see, e.g., FIG. 3B) of the second user 106. The computer instructions instruct the processor to compare the data of the beta waves, alpha waves, theta waves, delta waves, gamma waves, and epsilon waves of the first user 103 with the data of the beta waves, alpha waves, theta waves, delta waves, gamma waves, and epsilon waves of the second user 106. The computer instructions instruct the processor to identify the occurrence of neural synchrony between the first user 103 and the second user 106 based on the comparison of the beta waves, alpha waves, theta waves, delta waves, gamma waves, and epsilon waves of the first user 103 with the beta waves, alpha waves, theta waves, delta waves, gamma waves, and epsilon waves of the second user 106.

Referring again to FIGS. 1 and 2, a first monitoring device 115 is configured to detect pulse rate, oxygen saturation, cardiac cycle, and/or respiration rate of the first user 103. A second monitoring device 116 is configured to detect pulse rate, oxygen saturation, cardiac cycle, and/or respiration rate of the second user 106. The first monitoring device 115 and the second monitoring device 116 are in communication with the computer 108 to deliver data from the first monitoring device 115 and the second monitoring device 116 to the computer 108.

In an aspect of the present disclosure, each of the first monitoring device 115 and the second monitoring device 116 includes a pulse oximeter, an electrocardiography device, a photoplethysmography device, and/or a breath monitoring device.

Referring particularly to FIG. 4, behavioral synchronization may occur in a manner and with a pattern similar to that of neural synchrony (e.g., in-phase or phase-lagged).

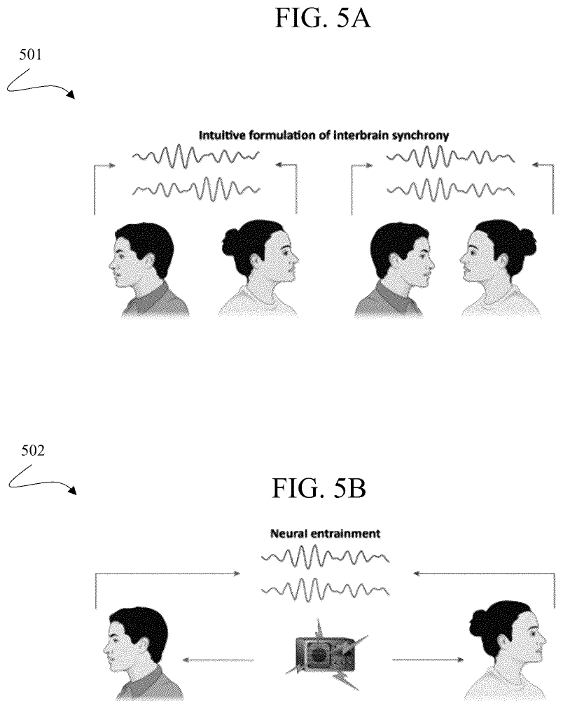

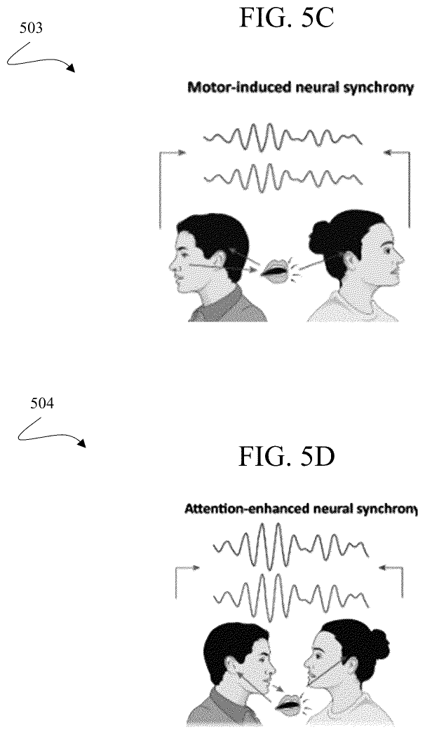

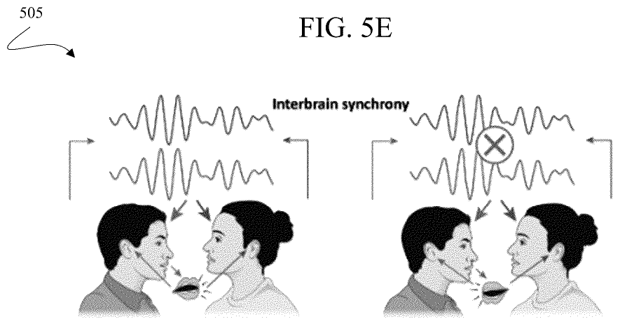

Referring particularly to FIGS. 5A to 5E, in an aspect of the present disclosure, the computer 108 is instructed to identify intuitive interbrain synchrony 501, neural entrainment 502, motor-induced neural synchrony 503, attention enhanced neural synchrony 504, and/or stimuli-based interbrain synchrony 505.

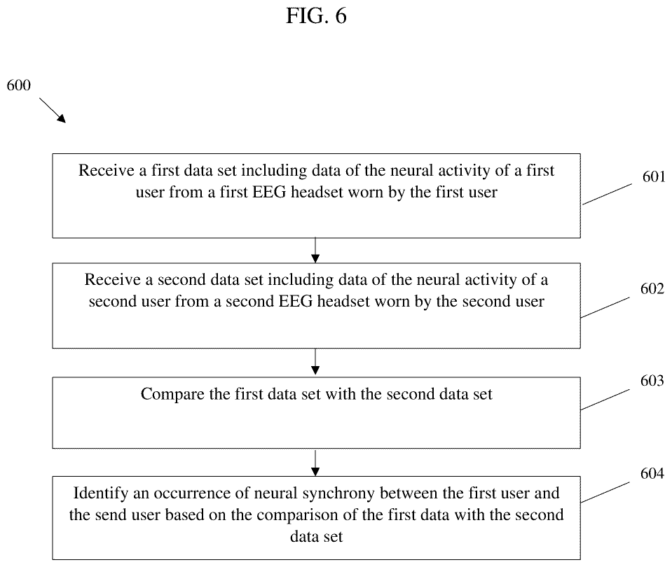

Referring particularly to FIG. 6, a computer-implemented method of identifying neural synchrony 600 includes receiving a first data set from a first EEG headset including a number of first electrodes 601. The first EEG headset is configured to be worn by a first user. The first electrodes of the first EEG headset are configured to detect neural activity in the first user. The first data set includes data of the neural activity of the first user. The computer-implemented method includes receiving a second data set from a second EEG headset including a number of second electrodes 602. The second EEG headset is configured to be worn by a second user. The second electrodes of the second EEG headset are configured to detect neural activity in the second user. The second data set includes data of the neural activity of the second user. The computer-implemented method includes comparing the first data set with the second data set 603. The computer-implemented method includes identifying an occurrence of neural synchrony between the first user and the second user based on the comparison of the first data with the second data set 604.

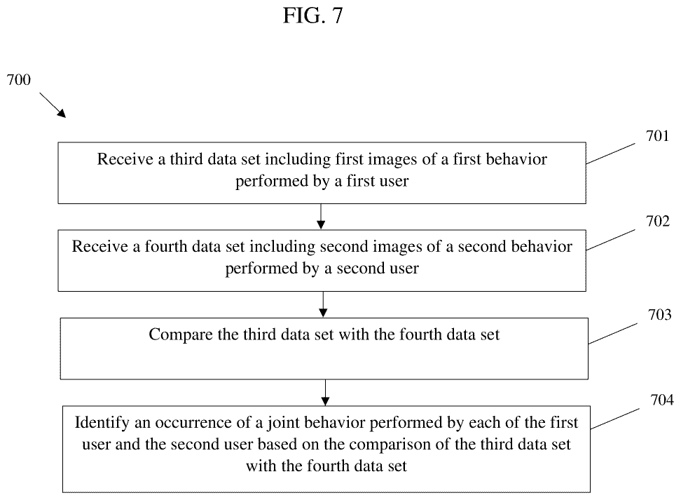

Referring particularly to FIG. 7, a computer-implemented method of identifying neural synchrony 700 includes receiving a third data set from the image capture device 701. The third data set includes data of images of a behavior performed by the first user. The method includes receiving a fourth data set from the image capture device 702. The fourth data set includes data of images of a second behavior performed by the second user. The method includes comparing the third data set with the fourth data set 703. The method includes identifying an occurrence of a joint behavior performed by each of the first user and the second user based on the comparison of the third data set with the fourth data set 704.

Referring particularly to FIG. 8, a general-purpose computer 800 is described. The computer (e.g., computer 108 described herein may have the same or substantially the same structure as the computer 800 or may incorporate at least some of the components of the computer 800). The general-purpose computer 800 can be employed to perform the various methods and algorithms described herein. The computer 800 may include a processor 801 connected to a computer-readable storage medium or a memory 802 which may be a volatile type memory, e.g., RAM, or a non-volatile type memory, e.g., flash media, disk media, etc. The processor 801 may be another type of processor such as, without limitation, a digital signal processor, a microprocessor, an ASIC, a graphics processing unit (GPU), field-programmable gate array (FPGA), or a central processing unit (CPU).

In some aspects of the disclosure, the memory 802 can be random access memory, read-only memory, magnetic disk memory, solid state memory, optical disc memory, and/or another type of memory. The memory 802 can communicate with the processor 801 through communication buses 803 of a circuit board and/or through communication cables such as serial ATA cables or other types of cables. The memory 802 includes computer-readable instructions that are executable by the processor 801 to operate the computer 800 to execute the algorithms described herein. The computer 800 may include a network interface 804 to communicate (e.g., through a wired or wireless connection) with other computers or a server. A storage device 805 may be used for storing data. The computer 800 may include one or more FPGAs 806. The FPGA 806 may be used for executing various machine learning algorithms. A display 807 may be employed to display data processed by the computer.

The computer 800 may employ one or more machine learning models or algorithms or a form of artificial intelligence to compare the data sets described herein for identifying a presence and/or monitoring an occurrence of neural synchrony. For example, the computer 800 may employ a classifier for comparing the data sets described herein for identifying and/or monitoring the present of neural synchrony.

The classifier may include a convolutional neural network (CNN, or ConvNet), a Bayesian network, a neural tree network, or a support-vector machine (SVM).

While a CNN may be employed, as described herein, other classifiers or machine learning models may similarly be employed. The machine learning model may be trained on tagged data, such as previously determined occurrences of neural synchrony. The trained CNN, trained machine learning model, or other form of decision or classification processes can be used to implement one or more of the methods, functions, processes, algorithms, or operations described herein. A neural network or deep learning model can be characterized in the form of a data structure storing data representing a set of layers containing nodes, and connections between nodes in different layers are formed or created that operate on an input to provide a decision or value as an output (e.g., a presence of neural synchrony, as described herein). While the presence of neural synchrony may be determined in a binary fashion, a quantitative likelihood of neural synchrony occurring may also be determined. For example, a probability of neural synchrony occurring can be determined based on a scale of 1 to 100, with a score of 1 indicating a very low likelihood of neural synchrony, and a score of 100 indicting a near certainty that neural synchrony is occurring. The value of 1 to 100 may be identified and mapped or graphed over time, such as on a time interval measured in milliseconds, or on a time interval measured in minutes or seconds. A predetermined threshold may be set (e.g., an 80% likelihood that neural synchrony is occurring based on a determined value of 80 or higher on a scale of 1 to 100).

As an example, the map or graph of neural synchrony may be displayed to a user to allow biofeedback to be employed by the user(s) to achieve, accelerate, or maintain neural synchrony. For example, a parent may receive real-time feedback via biofeedback (e.g., via displays 201 and/or 203) that neural synchrony is being achieved and maintained with the child of the parent.

Machine learning can be employed to enable the analysis of data and assist in making decisions. To benefit from using machine learning, a machine learning algorithm is applied to a set of training data and labels to generate a “model” which represents what the application of the algorithm has “learned” from the training data. Each element (e.g., one or more parameters, variables, characteristics, or “features”) of the set of training data is associated with a label or annotation that defines how the element should be classified by the trained model. A machine learning model predicts a defined outcome based on a set of features of an observation. The machine learning model is built by being trained on a dataset which includes features and known outcomes. There are various types of machine learning algorithms, including linear models, support vector machines (SVM), Bayesian networks, neural tree networks, random forest, and/or XGBoost. A machine learning model may include a set of layers of connected neurons that operate to decide (e.g., a classification) regarding a sample of input data. When trained (e.g., the weights connecting neurons have converged and become stable or within an acceptable amount of variation), the model will operate on new input data to generate the correct label, classification, weight, or score as an output. Other suitable machine learning models may be similarly employed.

A process for measuring neural synchrony is described in the following, the entire contents of which are incorporated by reference herein.

Leong, Victoria & de Barbaro, Kaya & Wass, Sam. (2014). “A Pilot Study on Mother-Infant Neural Synchrony During Live Social Interactions.” 10.13140/RG.2.1.1474.4403. Ramos, Christine. (2023). “The Magic & Mysteries of Parent-Child Synchrony.” Midwifery Today.” Issue 145.

Turk, Elise, et al. “In sync with your child: The potential of parent-child electroencephalography in developmental research.” Developmental psychobiology 64.3 (2022): e22221.

An exemplary data analysis process for determining a presence of neural synchrony includes:

Pre-processing in which continuous EEG data is divided into 2-second-long epochs.

Manual artifact rejection for excessive motion or noise.

Data is included for analysis if >60% of epochs accepted (“Video” 13 pairs; “Live” 10 pairs; “Norm” 9 pairs; “Still” 8 pairs).

Epochs are corrected for slow motion artifacts, blinks and drifts using Probabilistic Amplitude Demodulation.

Corrected epochs are filtered into 5 EEG frequency bands (delta 1-3, theta 4-7, alpha 8-12, beta 13-25, gamma 25-40 Hz).

Filtered epochs are edge-cropped to remove filtering and demodulation artifacts.

Final epoch length=1.5 s.

Measurement of Neural Synchrony (“Phase-Locking”).

Phase Synchronisation Index (PSI) computed between L-L and R-R mother-infant electrodes for each epoch and freq band. (PSI=|ei(nθ1−mθ2)).

Statistical Boot-Strapping-Statistical distribution for “random coupling” estimated by shuffling EEG segments across time and participants.

PSI value for upper 10th-percentile=threshold (no delta).

Percentage of epochs over threshold computed.

The devices described herein (e.g., the first and/or second EEG headsets, image capture device (e.g., digital camera), and/or the sensors may communicate with the computer described herein through a wired or wireless connection. For example, the wireless connection may be a Bluetooth connection.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

1. A system for identifying neural synchrony during a biofeedback session, comprising:
a first electroencephalogram (EEG) headset including a plurality of first electrodes, wherein the first EEG headset is configured to be worn by a first user, and wherein the first electrodes of the plurality of first electrodes of the first EEG headset are configured to detect neural activity in the first user;
a second EEG headset including a plurality of second electrodes, wherein the second EEG headset is configured to be worn by a second user, and wherein the second electrodes of the plurality of second electrodes of the second EEG headset are configured to detect neural activity in the second user;
at least one image capture device, wherein the at least one image capture device is configured to capture first images of at least one first behavior performed by the first user and second images of at least one second behavior performed by the second user; and
a computer in communication with the first EEG headset, the second EEG headset, the at least one image capture device, wherein the computer includes at least one processor and at least one memory, wherein the at least one memory is configured to store computer instructions configured to instruct the at least one processor to:
receive a first data set from the first EEG headset, wherein the first data set includes data of the neural activity of the first user;
receive a second data set from the second EEG headset, wherein the second data set includes data of the neural activity of the second user;
compare the first data set with the second data set;
identify an occurrence of neural synchrony between the first user and the second user based on the comparison of the first data with the second data set, wherein identifying the occurrence of neural synchrony by the neural network includes determining a probability of neural synchrony occurring between the first user and the second user based on a quantitative score, wherein the quantitative score is compared, by the neural network, to a predetermined threshold score to determine the occurrence of neural synchrony between the first user and the second user and the probability of neural synchrony occurring between the first user and the second user;
transmit data of the occurrence of neural synchrony between the first user and the second user, wherein the transmitted data includes the probability of neural synchrony occurring between the first user and the second user; and
display a graph of the occurrence of neural synchrony between the first user and the second user on at least one display during the biofeedback session thereby allowing real-time biofeedback to be employed by at least one of the first user or the second user to achieve or maintain neural synchrony during the biofeedback session.

2. The system of claim 1, wherein the computer instructions are further configured to instruct the processor to:
receive a third data set from the at least one image capture device, wherein the third data set includes data of the first images of the at least one first behavior performed by the first user;
receive a fourth data set from the at least one image capture device, wherein the fourth data set includes data of the second images of the at least one second behavior performed by the second user;
compare the third data set with the fourth data set; and
identify an occurrence of a joint behavior performed by each of the first user and the second user based on the comparison of the third data set with the fourth data set.

3. The system of claim 1, wherein the first and second data sets are each captured during a same predetermined time period as each other, and wherein the first data set is compared with the second data set with respect to the same predetermined time period.

4. The system of claim 3, wherein the third and fourth data sets are each captured during the same predetermined time period, and wherein the third data set is compared with the fourth data set with respect to the same predetermined time period.

5. The system of claim 1, wherein the first EEG headset is configured to be worn by a child, and wherein the second EEG headset is configured to be worn by a parent of the child.

6. The system of claim 1, wherein the first electrodes of the plurality of first electrodes, or the second electrodes of the plurality of second electrodes are configured to capture at least one of beta waves, alpha waves, theta waves, delta waves, gamma waves, or epsilon waves.

7. The system of claim 6, wherein the first data set includes data of beta waves, alpha waves, theta waves, delta waves, gamma waves, and epsilon waves of the first user, wherein the second data set includes data of beta waves, alpha waves, theta waves, delta waves, gamma waves, and epsilon waves of the second user, and wherein the computer instructions are further configured to instruct the processor to:
compare the data of the beta waves, alpha waves, theta waves, delta waves, gamma waves, and epsilon waves of the first user with the data of the beta waves, alpha waves, theta waves, delta waves, gamma waves, and epsilon waves of the second user; and
identify the occurrence of neural synchrony between the first user and the second user based on the comparison of the beta waves, alpha waves, theta waves, delta waves, gamma waves, and epsilon waves of the first user with the beta waves, alpha waves, theta waves, delta waves, gamma waves, and epsilon waves of the second user.

8. (canceled)

9. The system of claim 1, wherein the image capture device is a digital camera.

10. The system of claim 1, further including:
at least one first display configured to receive the first data set from the first EEG headset, wherein the at least one first display is configured to display a first visual representation of brain wave data of the first user; and
at least one second display configured to receive the second data set from the second EEG headset, wherein the at least one second display is configured to display a second visual representation of brain wave data of the second user.

11. The system of claim 10, wherein the at least one first display is arranged to display the first visual representation of brain wave data to the first user, and wherein the at least one second display is arranged to display the second visual representation of brain wave data to the second user.

12. The system of claim 10, wherein the at least one first display is arranged to display the first visual representation of brain wave data to the second user, and wherein the at least one second display is arranged to display the second visual representation of brain wave data to the first user.

13. The system of claim 1, further including:
at least one first monitoring device configured to detect at least one of pulse rate, oxygen saturation, cardiac cycle, or respiration rate of the first user; and
at least one second monitoring device configured to detect at least one of pulse rate, oxygen saturation, cardiac cycle, or respiration rate of the second user, wherein the at least one first monitoring device and the at least one second monitoring device are each in communication with the computer to deliver data from the at least one first monitoring device and the at least one second monitoring device to the computer.

14. The system of claim 13, wherein each of the at least one first monitoring device and the at least one second monitoring device includes at least one of a pulse oximeter, a electrocardiography device, a photoplethysmography device, or a breath monitoring device.

15. A computer-implemented method of identifying neural synchrony during a biofeedback session, comprising:
receiving a first data set from a first electroencephalogram (EEG) headset including a plurality of first electrodes, wherein the first EEG headset is configured to be worn by a first user, and wherein the first electrodes of the plurality of first electrodes of the first EEG headset are configured to detect neural activity in the first user, wherein the first data set includes data of the neural activity of the first user;
receiving a second data set from a second EEG headset including a plurality of second electrodes, wherein the second EEG headset is configured to be worn by a second user, and wherein the second electrodes of the plurality of second electrodes of the second EEG headset are configured to detect neural activity in the second user, wherein the second data set includes data of the neural activity of the second user;
comparing the first data set with the second data set;
identifying an occurrence of neural synchrony between the first user and the second user based on the comparison of the first data with the second data set, wherein identifying the occurrence of neural synchrony includes determining a probability of neural synchrony occurring between the first user and the second user based on a quantitative score, wherein the quantitative score is compared to a predetermined threshold score to determine the occurrence of neural synchrony between the first user and the second user and the probability of neural synchrony occurring between the first user and the second user;
transmitting data of the occurrence of neural synchrony between the first user and the second user, wherein the transmitted data includes the probability of neural synchrony occurring between the first user and the second user; and
displaying a graph of the occurrence of neural synchrony between the first user and the second user on at least one display during the biofeedback session thereby allowing real-time biofeedback to be employed by at least one of the first user or the second user to achieve or maintain neural synchrony during the biofeedback session.

16. The computer-implemented method of claim 15, further including:
receiving a third data set from the at least one image capture device, wherein the third data set includes data of the first images of the at least one first behavior performed by the first user;
receiving a fourth data set from the at least one image capture device, wherein the fourth data set includes data of the second images of the at least one second behavior performed by the second user;
comparing the third data set with the fourth data set; and
identifying an occurrence of a joint behavior performed by each of the first user and the second user based on the comparison of the third data set with the fourth data set.

17. The computer-implemented method of claim 16, wherein the first data set includes data of beta waves, alpha waves, theta waves, delta waves, gamma waves, and epsilon waves of the first user, wherein the second data set includes data of beta waves, alpha waves, theta waves, delta waves, gamma waves, and epsilon waves of the second user, and wherein the computer-implemented method further includes:
comparing the data of the beta waves, alpha waves, theta waves, delta waves, gamma waves, and epsilon waves of the first user with the data of the beta waves, alpha waves, theta waves, delta waves, gamma waves, and epsilon waves of the second user; and
identifying the occurrence of neural synchrony between the first user and the second user based on the comparison of the beta waves, alpha waves, theta waves, delta waves, gamma waves, and epsilon waves of the first user with the beta waves, alpha waves, theta waves, delta waves, gamma waves, and epsilon waves of the second user.

18. The computer-implemented method of claim 15, further including:
receiving, by at least one first display, the first data set from the first EEG headset, wherein the at least one first display displays a first visual representation of brain wave data of the first user; and
receiving, by at least one second display, the second data set from the second EEG headset, wherein the at least one second display displays a second visual representation of brain wave data of the second user.

19. The computer-implemented method of claim 18, wherein the at least one first display displays the first visual representation of brain wave data to the first user, and wherein the at least one second display displays the second visual representation of brain wave data to the second user.

20. The computer-implemented method of claim 18, wherein the at least one first display displays the first visual representation of brain wave data to the second user, and wherein the at least one second display displays the second visual representation of brain wave data to the first user.
